(19) United States
(12) Patent Application Publication
(10) Pub. No.: US 2003/0147840 A1
(43) Pub. Date: Aug. 7, 2003

(54) Reducing composition for treating keratin fibres, comprising a particular aminosilicone

(76) Inventors: Frederic Legrand, Courbevoie (FR); Jean-Marie Millequant, Saint-Maur des Fosses (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/290,192

(22) Filed: Nov. 8, 2002

(30) Foreign Application Priority Data
Nov. 8, 2001 (FR) 01 14473

(51) Int. Cl. A61K 7/135; A61K 7/06; A61K 7/11

(52) U.S. Cl. 424/70.122; 424/62

(57) ABSTRACT

A cosmetic composition for treating human keratin fibres, such as hair, comprising, in a cosmetically acceptable medium:

(i) at least one reducing agent, and

(ii) at least one aminosilicone comprising at least one aminoethylimino(C₄-C₈)alkyl group.

Used for bleaching and permanently reshaping human keratin fibres. Processes, kits and devices for bleaching or permanently reshaping human keratin fibres using the composition.

The present disclosure relates to a novel composition for treating human keratin fibres, such as hair, comprising, in a cosmetically acceptable medium:

(i) at least one reducing agent, and

(ii) at least one aminosilicone comprising at least one aminoethylimino(C₄-C₈)alkyl group.

This disclosure also relates to the uses of the said composition for bleaching and permanently reshaping human keratin fibres and to bleaching and permanent-reshaping processes and devices using the said composition.

It is known practice to bleach keratin fibres, such as human hair, with bleaching compositions comprising at least one oxidizing agent. Among the oxidizing agents conventionally used, mention may be made of hydrogen peroxide and compounds capable of producing hydrogen peroxide by hydrolysis, such as urea peroxide and persalts such as perborates, percarbonates, and persulphates, such as, for example, hydrogen peroxide and persulphates.

However, it is also known practice to bleach human keratin fibres such as the hair. For example, hair may be artificially dyed with exogenous colorants, using reducing agents such as ascorbic acid or thiols, for example, cysteine.

It is also known practice to permanently reshape human keratin fibres, such as hair. Such reshaping is done by example by applying compositions comprising one or more reducing agents to the hair. For example, the hair may be placed under tension beforehand using mechanical devices such as curlers. The hair is thus reduced and then reoxidized in the desired shape, usually after rinsing. Not only may atmospheric oxygen be used, but also an oxidizing agent that may be chosen from aqueous hydrogen peroxide solution and alkali metal bromates may be used.

The reducing agents that may be used for the permanent reshaping of hair include thiols such as thioglycolic acid, its salts and its esters, thiolactic acid and its salts, cysteine or cysteamine, and sulphites.

Compositions for bleaching hair using reducing agents may be in the form of ready-to-use compositions comprising anhydrous products (powders or creams) comprising at least one the reducing agent, which may be mixed at the time of use with an aqueous composition optionally comprising a pH agent. Bleaching compositions may also be in the form of ready-to-use aqueous compositions comprising the at least one reducing agent at the appropriate pH.

Reducing compositions for permanently reshaping the hair may generally be the form of ready-to-use aqueous compositions or in the form of pulverulent or liquid anhydrous compositions that are mixed at the time of use with an aqueous composition at the appropriate pH.

Moreover, it is known that the treatment for permanently reshaping the hair, and, for example, the bleaching treatment, can often be aggressive and result in poor cosmetic properties of the hair, such as difficult disentangling, an unpleasant feel, or coarse, dull hair, or alternatively hair charged with static electricity, and in degradation of the fibres. After considerable research conducted in this matter, the inventors have discovered that by using at least one aminosilicone defined below in a reducing composition for bleaching and/or permanently reshaping human keratin fibres, such as hair, at least one of these drawbacks may be overcome, with conditioning and remanent effects that can be superior to those of the systems previously used, without, however, impairing at least one of the intensity and homogeneity of the bleaching or permanent-reshaping results.

Thus, the condition of the human keratin fibres may be improved and the said fibres may maintain their soft feel, their ease of disentangling and their sheen after shampooing several times.

The phrase “improvement in the condition of the fibre” means a reduction in the porosity or the alkaline solubility of the fibre and an improvement in at least one cosmetic property such as, for example, in the smoothness, softness and ease of disentangling and of styling.

This effect can be remanent, i.e. long-lasting.

The porosity is measured by fixing, at 37° C. and at pH 10, for 2 minutes, 2-nitro-para-phenylenediamine at 0.25% in an ethanol/pH 10 buffer mixture (10/90 volume ratio).

The alkaline solubility corresponds to the loss of mass of a sample of 100 mg of keratin fibres under the action of decinormal sodium hydroxide for 30 minutes at 65° C.

These discoveries form at least a portion of the basis for at least one embodiment disclosed herein.

One new embodiment therefore relates to a cosmetic composition for treating human keratin fibres, such as hair, comprising, in a cosmetically acceptable medium:

(i) at least one reducing agent, and

(ii) at least one aminosilicone comprising at least one aminoethylimino(C₄-C₈)alkyl group.

Another new embodiment relates to the use of a ready-to-use composition for bleaching or permanently reshaping human keratin fibres, such as hair, comprising at least one reducing agent in a medium that is suitable for bleaching or permanent reshaping and at least one aminosilicone comprising at least one aminoethylimino(C₄-C₈)alkyl group.

The phrase “ready-to-use composition” means the composition is intended to be applied as it is in unmodified form to the human keratin fibres, such as hair, i.e. the composition may be stored in unmodified form before use or may result from the extemporaneous mixing of at least two or more compositions.

When the ready-to-use composition results from the extemporaneous mixing of at least two compositions, the at least one aminosilicone, defined herein, is present in at least one pre-mixed composition.

As such, the at least one aminosilicone defined herein may be present in an anhydrous composition in the form of a powder, for example, pulverulent powder, or in the form of a cream and/or in at least one aqueous composition.

The at least one aminosilicone defined herein may also be present in at least one aqueous composition, which may be mixed at the time of use with a composition that can be either aqueous or anhydrous in the form of a powder or a cream and comprising at least one reducing agent.

The composition may be a single composition comprising at least one reducing agent and at least one aminosilicone as defined herein.

The at least one aminosilicone defined herein can be used in an anhydrous composition further comprising at least one reducing agent, the composition, for example, being intended to be diluted before being applied to the fibres.

This disclosure also relates to a bleaching process and/or a permanent-reshaping process for human keratin fibres, such as hair, using the ready-to-use bleaching or permanent-reshaping composition as described herein. The application of the composition may be followed, in the case of permanent reshaping, by the application, optionally after rinsing, of an oxidizing composition.

This disclosure relates further to bleaching devices or packaging “kits” comprising a ready-to-use composition.

In an embodiment, a multi-compartment kit or device comprises a first compartment comprising at least one anhydrous powder or an anhydrous cream or an aqueous composition, and the second compartment comprising an aqueous composition, at least one of the two compartments comprising at least one reducing agent and at least one of the two compartments comprising at least one aminosilicone defined herein.

At least one other characteristic, aspect, subject and advantage of embodiments disclosed herein may emerge even more clearly on reading the description and the examples that follow without however exhibiting a limiting character.

Aminosilicones

The at least one aminosilicone may, for example, have the following formula:

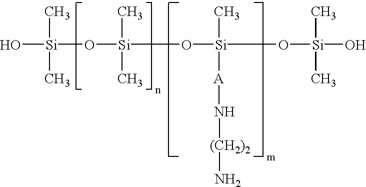

wherein:

A is chosen from linear and branched C₄-C₈ alkylene radicals, for example, C₄ alkylene radicals and

m and n are numbers such that the sum (n+m) can range, for example, from 1 to 2 000 and further, for example, from 50 to 150, n may be a number ranging from 0 to 1 999, for example, from 49 to 149, and m may be a number ranging from 1 to 2 000, for example, from 1 to 10.

The term “alkylene radical” means divalent saturated hydrocarbon-based groups.

The viscosity of the at least one aminosilicone, for example, can be greater than 25 000 mm²/s at 25° C.

For example, this viscosity may range from 30 000 to 200 000 mm²/s at 25° C. and further, for example, from 30 000 to 150 000 mm²/s at 25° C.

The viscosity of the at least one aminosilicone is measured at 25° C. according to the standard “ASTM 445 Appendix C”.

The at least one aminosilicone has a weight-average molecular mass ranging, for example, from 2000 to 1 000 000 and further, for example, from 3500 to 200 000.

The weight-average molecular masses of the at least one aminosilicone is measured by Gel Permeation Chromatography (GPC) at room temperature, as polystyrene equivalents. The columns used are styragel μ columns. The eluent is THF, and the flow rate is 1 ml/minute. 200 μl of a solution at 0.5% by weight of silicone in THF are injected. The detection is performed by refractometry and UV-metry.

When at least one aminosilicone is used, one new embodiment involves using the at least one aminosilicone in the form of an oil-in-water emulsion. The oil-in-water emulsion may comprise at least one surfactant. The at least one surfactant may be of any nature, for example, cationic and/or nonionic.

The silicone particles in the emulsion have a mean size ranging, for example, from 3 to 500 nanometres, further, for example, from 5 to 300 nanometers, further, for example, from 10 to 275 nanometers and even further, for example, from 150 to 275 nanometers. Such particle sizes are measured with a laser granulometer.

An example of a silicone corresponding to this formula is DC2-8299® from the company Dow Corning.

The at least one aminosilicone may be used in the reducing composition in an amount ranging from 0.01% to 20% by weight relative to the total weight of the composition. For example, this amount may range from 0.1% to 15% by weight and even further, for example, from 0.5% to 10% by weight relative to the total weight of the composition.

Reducing Agents

The at least one reducing agent that may be used may be chosen, for example, from the group formed by thiols, such as cysteine, thioglycolic acid, thiolactic acid, salts thereof and esters thereof, cysteamine and its salts, and sulphites.

According to one embodiment, ascorbic acid, its salts and its esters, erythorbic acid, its salts and its esters, and sulphinates, for example, sodium hydroxymethanesulphinate, may also be used.

The at least one reducing agent can be used in a composition in concentrations ranging from about 0.1% to 30%, for example, from about 0.5% to 20% by weight, relative to the total weight of the composition.

Ingredients

The compositions may also comprise agents for adjusting the rheology such as those chosen from cellulosic thickeners (hydroxyethylcellulose, hydroxypropylcellulose, carboxymethylcellulose, etc.), guar gum and its derivatives (hydroxypropyl guar, etc.), gums of microbial origin (xanthan gum, scleroglucan gum, etc.), and synthetic thickeners such as crosslinked homopolymers of acrylic acid or of acrylamidopropanesulphonic acid.

A composition may also comprise at least one ionic or nonionic associative polymer chosen, for example, from the polymers sold under the names Pemulen® TR1 and TR2 by the company Goodrich, Salcare SC 90® by the company Allied Colloids, Aculyn® 22, 28, 33, 44 or 46 by the company Rohm & Haas, and Elfacos® T210 and T212 by the company Akzo, in a proportion ranging from 0.01% to 10% by weight relative to the total weight of the composition.

The composition may also comprise at least one cationic or amphoteric polymer that is well-known in the art of dyeing human keratin fibres, in an amount ranging from 0.01% to 10% by weight, for example, from 0.05% to 5% and further, for example, from 0.1% to 3% by weight, relative to the total weight of the composition.

Cationic Polymers

The expression “cationic polymer” means any polymer comprising cationic groups and/or groups which may be ionized into cationic groups.

The at least one cationic polymers which may be used, may be chosen from any of those already known by those skilled in the art as improving at least one cosmetic property of the hair, for example, those described in European patent application EP-A-337 354 and in French Patent Nos. FR-2 270 846, 2 383 660, 2 598 611, 2 470 596 and 2 519 863.

The at least one cationic polymer may, for example be chosen from those comprising units comprising at least one amine group chosen from primary, secondary, tertiary and quaternary amine groups, which may either form part of the main polymer chain or may be borne by a side substituent directly attached to the main polymer chain.

The at least one cationic polymer used may have a number-average molecular mass ranging from 500 to 5×10⁶ and further, for example, from 10³ and 3×10⁶.

Among the at least one cationic polymer which may be mentioned are those chosen from polymers of polyamine, polymers of polyamino amide and polymers of polyquaternary ammonium. These polymers are known in the art.

The polymers of polyamine, polymers of polyamino amide and polymers of polyquaternary ammonium that may be used are described, for example, in French patent Nos. 2 505 348 and 2 542 997. Among the cationic polymers, mention may be made of:

(1) homopolymers or copolymers derived from acrylic or methacrylic esters or amides and comprising at least one unit chosen from units of formula (I), (II), (III) and (IV) below:

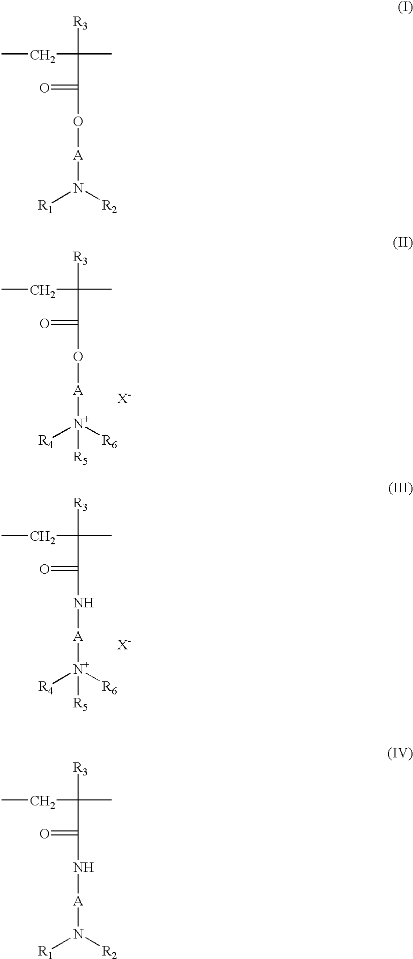

wherein:

R₃, which may be identical or different, is chosen from a hydrogen atom and a CH₃ radical;

A, which may be identical or different, is chosen from a linear and branched alkyl groups of 1 to 6 carbon atoms, such as 2 or 3 carbon atoms, and hydroxyalkyl groups of 1 to 4 carbon atoms;

R₄, R₅ and R₆, which may be identical or different, are chosen from alkyl groups comprising from 1 to 18 carbon atoms, for example, an alkyl groups comprising from 1 to 6 carbon atoms and benzyl radicals;

R₁ and R₂, which may be identical or different, are chosen from hydrogen and alkyl groups comprising from 1 to 6 carbon atoms, for example, methyl and ethyl groups;

X⁻ is an anion derived from an inorganic or organic acid, such as a methosulphate anion or an anion chosen from halides such as chloride and bromide.

The polymers of family (1) can also comprise at least one unit derived from comonomers that may be chosen from the family of acrylamides, methacrylamides, diacetoneacrylamides, acrylamides and methacrylamides substituted on the nitrogen with at least one group chosen from lower (C₁-C₄) alkyls, acrylic and methacrylic acids, and esters thereof, and vinyllactams, such as vinylpyrrolidone, and vinylcaprolactam, and vinyl esters.

Thus, among these polymers of family (1), mention may be made of:

the copolymers of acrylamide and of dimethylaminoethyl methacrylate quaternized with dimethyl sulphate or with a dimethyl halide, such as the product sold under the name Hercofloc™ by the company Hercules,

copolymers of acrylamide and of methacryloyloxyethyltrimethylammonium chloride described, for example, in patent application EP-A-080 976 and sold under the name Bina Quat P 100 by the company Ciba Geigy,

the copolymer of acrylamide and of methacryloyloxyethyltrimethylammonium methosulphate sold under the name Reten by the company Hercules,

quaternized or non-quaternized vinylpyrrolidone/dialkylaminoalkyl acrylate or methacrylate copolymers, such as the products sold under the name “Gafquat” by the company ISP, for example, “Gafquat 734” or “Gafquat 755,” or the products known as “Copolymer 845, 958 and 937.” These polymers are described in detail in French Patent Nos. 2 077 143 and 2 393 573,

dimethylaminoethyl methacrylate/vinylcaprolactam/vinylpyrrolidone terpolymers, such as the product sold under the name Gaffix VC 713 by the company ISP,

vinylpyrrolidone/methacrylamidopropyldimethylamine copolymers sold, for example, under the name Styleze CC 10 by ISP, and

quaternized vinylpyrrolidone/dimethylaminopropylmethacrylamide copolymers such as the product sold under the name “Gafquat HS 100” by the company ISP.

(2) The cellulose ether derivatives comprising quaternary ammonium groups, described in French Patent No. 1 492 597, and the polymers sold under the names “JR” (JR 400, JR 125 and JR 30M) or “LR” (LR 400, or LR 30M) by the company Union Carbide Corporation. These polymers are also defined in the CTFA dictionary as quaternary ammoniums of hydroxyethylcellulose which has reacted with an epoxide substituted with a trimethylammonium group.

(3) The cationic cellulose derivatives such as cellulose copolymers or cellulose derivatives grafted with a water-soluble monomer of quaternary ammonium, and described, for example, in U.S. Pat. No. 4,131,576, such as hydroxyalkylcelluloses, for instance hydroxymethylcelluloses, hydroxyethylcelluloses and hydroxypropylcelluloses grafted, for example, with a salt chosen from methacryloyloxyethyltrimethylammonium salts, methacrylamidopropyltrimethylammonium salts and dimethyldiallylammonium salts.

The commercial products corresponding to this definition are, for example, the products sold under the names “Celquat L 200” and “Celquat H 100” by the company National Starch.

(4) The cationic polysaccharides described in U.S. Pat. Nos. 3,589,578 and 4,031,307, such as guar gums comprising cationic trialkylammonium groups. For example, guar gums modified with a salt (e.g. chloride) of 2,3-epoxypropyltrimethylammonium may also be used.

Such products are sold, for example, under the trade names Jaguar C13 S, Jaguar C 15, Jaguar C 17 or Jaguar C162 by the company Meyhall.

(5) Polymers comprising piperazinyl units and divalent alkylene or hydroxyalkylene radicals comprising straight or branched chains, optionally interrupted by at least one atom chosen from oxygen, sulphur and nitrogen atom or by at least one aromatic or heterocyclic ring, and also at least one oxidation and/or quaternization product of these polymers. Such polymers are described, for example, in French Patent Nos. 2 162 025 and 2 280 361.

(6) Water-soluble polyamino amides prepared, for example, by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative. The crosslinking agent can be used in proportions ranging from 0.025 to 0.35 mol per amine group of the polyamino amide. These polyamino amides can be alkylated or, if they comprise at least one tertiary amine functions, they can be quaternized. Such polymers are described, for example, in French Patent Nos. 2 252 840 and 2 368 508.

(7) The polyamino amide derivatives resulting from the condensation of polyalkylene polyamines with polycarboxylic acids followed by alkylation with difunctional agents. Mention may be made, for example, of adipic acid/dialkylaminohydroxyalkyldialkylene-triamine polymers in which the alkyl radical comprises from 1 to 4 carbon atoms and, for example, may be chosen from methyl, ethyl and propyl groups. Such polymers are described in particular in French Patent No. 1 583 363.

Among these derivatives, mention may be made, for example, of the adipic acid/dimethylaminohydroxypropyl/diethylenetriamine polymers sold under the name “Cartaretine F, F4 or F8” by the company Sandoz.

(8) The polymers obtained by reaction of a polyalkylene polyamine comprising two primary amine groups and at least one secondary amine group with a dicarboxylic acid chosen from diglycolic acid and saturated aliphatic dicarboxylic acids comprising from 3 to 8 carbon atoms. The molar ratio between the polyalkylene polyamine and the dicarboxylic acid may range, for example, from 0.8:1 to 1.4:1; the polyamino amide resulting therefrom may be reacted with epichlorohydrin in a molar ratio of epichlorohydrin relative to the secondary amine group of the polyamino amide ranging from 0.5:1 and 1.8:1. Such polymers are described, for example, in U.S. Pat. Nos. 3,227,615 and 2,961,347.

Other non-limiting examples of such derivatives include the adipic acid/epoxypropyl/diethylenetriamine copolymers, for example, sold under the name “Hercosett 57” by the company Hercules Inc. or under the name “PD 170” or “Delsette 101” by the company Hercules Inc..

(9) Cyclopolymers of alkyldiallylamine or of dialkyldiallylammonium, such as the homopolymers and copolymers comprising, as main constituent of the chain, at least one unit chosen from formulae (V) and (VI):

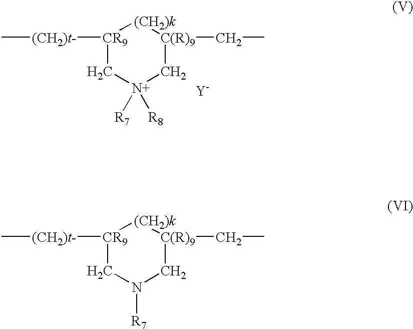

wherein k and t are equal to 0 or 1, the sum k+t being equal to 1;

R₉ is chosen from a hydrogen atom and a methyl radical;

R₇ and R₈, which may be identical or different, are chosen from alkyl groups comprising from 1 to 8 carbon atoms, hydroxyalkyl groups in which the alkyl group, for example, comprises from 1 to 5 carbon atoms, and lower C₁-C₄ amidoalkyl groups, or R₇ and R₈ can be chosen from, together with the nitrogen atom to which they are attached, heterocyclic groups such as piperidyl and morpholinyl; in one new embodiment, R₇ and R₈, which may be identical or different are chosen from alkyl groups comprising from 1 to 4 carbon atoms;

Y⁻ is an anion chosen from bromide, chloride, acetate, borate, citrate, tartrate, bisulphate, bisulphite, sulphate and phosphate. These polymers are described, for example, in French Patent No. 2 080 759 and in its Certificate of Addition 2 190 406.

Among the polymers defined above, mention may be made, for example, of the dimethyldiallylammonium chloride homopolymer sold under the name “Merquat 100” by the company Calgon (and its homologues of low weight-average molecular mass) and copolymers of diallyldimethylammonium chloride and of acrylamide, sold under the name “Merquat 550.”

(10) The quaternary diammonium polymer comprising repeating units corresponding to the formula:

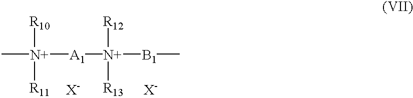

wherein:

R₁₀, R₁₁, R₁₂ and R₁₃, which may be identical or different, are chosen from aliphatic radicals, alicyclic radicals, and arylaliphatic radicals comprising from 1 to 20 carbon atoms and from lower hydroxyalkylaliphatic radicals, or R₁₀, R₁₁, R₁₂ and R₁₃, together or separately, constitute, with the nitrogen atoms to which they are attached, heterocycles optionally comprising a second hetero atom other than nitrogen, or R₁₀, R₁₁, R₁₂ and R₁₃, which may be identical or different, are chosen from linear and branched C₁-C₆ alkyl radicals substituted with at least one group chosen from nitrile, ester, acyl and amide groups and groups of formulae —CO—O—R₁₄—D and —CO—NH—R₁₄—D wherein R₁₄ is chosen from alkylene groups and D is chosen from quaternary ammonium groups;

A₁ and B₁, which may be identical or different, are chosen from linear and branched, saturated and unsaturated polymethylene groups comprising from 2 to 20 carbon atoms. The polymethylene groups may comprise, linked to or intercalated in the main chain, at least one entity chosen from aromatic rings, oxygen, and sulphur atoms, and sulphoxide, sulphone, disulphide, amino, alkylamino, hydroxyl, quaternary ammonium, ureido, amide and ester groups,

X⁻ is an anion chosen from anions derived from mineral and organic acids;

A₁, R₁₀ and R₁₂ may optionally form, with the two nitrogen atoms to which they are attached, a piperazine ring. In addition, if A₁ is a radical chosen from linear and branched, saturated and unsaturated alkylene and hydroxyalkylene radicals, B₁ can also be chosen from a group —(CH₂)ₙ—CO—D—OC—(CH₂)ₙ— wherein n ranges from 1 and 100, such as from 1 to 50.

D is chosen from:

a) a glycol residue of formula: —O—Z—O—, wherein Z is chosen from linear and branched hydrocarbon-based radicals and a group corresponding to one of the following formulae:

—(CH₂—CH₂—O)ₓ—CH₂—CH₂— and

—[CH₂—CH(CH₃)—O]_{y}—CH₂—CH(CH₃)—

wherein x and y, which may be identical or different, are each an integer ranging from 1 to 4, representing a defined and unique degree of polymerization or any number ranging from 1 to 4 representing an average degree of polymerization;

b) a bis-secondary diamine residue such as a piperazine derivative;

c) a bis-primary diamine residue of formula: —NH—Y—NH—, wherein Y is chosen from a linear and branched hydrocarbon-based radicals, and the divalent radical —CH₂—CH₂—S—S—CH₂—CH₂—; and

d) a ureylene group of formula: —NH—CO—NH—

In one embodiment, X⁻ is an anion such as chloride or bromide.

These polymers may have a number-average molecular mass ranging from 1000 to 100 000.

These polymers are described, for example, in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Further, the polymers that comprise repeating units corresponding to formula (VIII) below can be used:

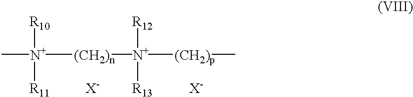

wherein R₁₀, R₁₁, R₁₂ and R₁₃, which may be identical or different, are chosen from alkyl and hydroxyalkyl radicals comprising from 1 to 4 carbon atoms, n and p, which may be identical or different, are integers ranging from 2 to 20, and X₋ is an anion chosen from anions derived from mineral and organic acids, for example where R₁₀, R₁₁, and R₁₃ are each a methyl radical, n=3, p=6, and X=Cl, known as hexadimethine chloride, according to the INCI (CFTA) nomenclature.

(11) Polyquaternary ammonium polymers comprising repeating units of formula (IX):

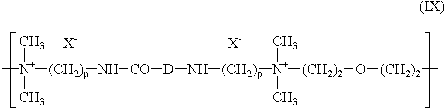

wherein p is an integer ranging from 1 to 6, D may be nothing or may represent a group—(CH₂)ᵣ—CO— in which r may be an integer ranging from 4 to 7, and X⁻ is an anion.

Such polymers may be prepared according to the processes described in U.S. Pat. Nos. 4,157,388, 4,702,906 and 4,719,282. They are described, for example, in European patent application EP-A-122 324.

Among these polymers, mention may be made, for example, of “Mirapol A 15”, “Mirapol AD1”, “Mirapol AZ1” and “Mirapol 175” sold by the company Miranol.

(12) Quaternary polymers of vinylpyrrolidone and of vinylimidazole, such as the products sold under the names Luviquat FC 905, FC 550 and FC 370 by the company BASF.

(13) Polyamines such as Polyquart H sold by Henkel, under the reference name “Polyethylene glycol (15) tallow polyamine” in the CTFA dictionary.

(14) Crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising olefinic unsaturation, such as methylenebisacrylamide. A crosslinked acrylamide/methacryloyloxyethyltrimethylammonium chloride copolymer (20/80 by weight) in the form of a dispersion comprising 50% by weight of the copolymer in mineral oil can be used. This dispersion is sold under the name “Salcare® ( SC 92” by the company Allied Colloids. In another embodiment, a crosslinked methacryloyloxyethyltrimethylammonium chloride homopolymer comprising about 50% by weight of the homopolymer in mineral oil or in a liquid ester can also be used. These dispersions are sold under the names “Salcare® SC 95” and “Salcare® SC 96” by the company Allied Colloids.

Other cationic polymers which can be used are chosen from polyalkyleneimines, for example, polyethyleneimines, polymers comprising vinylpyridine and vinylpyridinium units, condensates of polyamines and of epichlorohydrin, quaternary polyureylenes and chitin derivatives.

Among all the cationic polymers that may be used, non-limiting examples include the polymers of families (1), (9), (10), (11) and (14) and, in one new embodiment, polymers comprising repeating units chosen from units of formulae (W) and (U) below can be used:

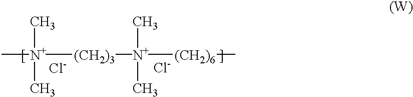

and, for example, those polymers comprising repeating units of formula (W) whose weight-average molar mass, determined by gel permeation chromatography ranges from 9500 to 9900;

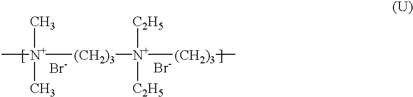

and, for example, those polymers comprising repeating units of formula (U) whose weight-average molar mass, determined by gel permeation chromatography, is about 1200.

The concentration of the at least one cationic polymer in the composition may range, for example, from 0.01% to 10% by weight relative to the total weight of the composition, for example from 0.05% to 5% by weight and even further, for example, from 0.1% to 3% by weight relative to the total weight of the composition.

Amphoteric polymers

The amphoteric polymers which may be used can be chosen from polymers comprising units K and M randomly distributed in the polymer chain, wherein K is a unit derived from a monomer comprising at least one basic nitrogen atom and M is a unit derived from an acidic monomer comprising at least one carboxylic or sulphonic groups, or K and M may be chosen from groups derived from zwitterionic carboxybetaine or sulphobetaine monomers.

K and M may also be chosen from a cationic polymer chain comprising at least one group chosen from primary, secondary, tertiary and quaternary amine groups, in which at least one of the amine groups bears a carboxylic or sulphonic group linked via a hydrocarbon-based radical, or K and M can form part of a chain of a polymer comprising an α,β-dicarboxylic ethylene unit in which one of the carboxylic groups has been made to react with a polyamine comprising at least one amine chosen from primary and secondary amine groups.

The amphoteric polymers corresponding to the above definition may be chosen from the following polymers:

(1) polymers resulting from the copolymerization of at least one monomer derived from a vinyl compound bearing a carboxylic group such as acrylic acid, methacrylic acid, maleic acid, α-chloroacrylic acid, and at least one basic monomer derived from a substituted vinyl compound comprising at least one basic atom, such as dialkylaminoalkyl methacrylate and acrylate, dialkylaminoalkylmethacrylamide and -acrylamide. Such compounds are described, for example, in U.S. Pat. No. 3,836,537.

Mention may also be made of the sodium acrylate/acrylamidopropyltrimethylammonium chloride copolymer sold under the name Polyquart KE 3033 by the company Henkel.

The vinyl compound may also be a dialkyldiallylammonium salt such as dimethyldiallylammonium chloride. The copolymers of acrylic acid and of the latter monomer are sold under the names “Merquat 280,” “Merquat 295” and “Merquat Plus 3330” by the company Calgon.

(2) polymers comprising units derived from:

a) at least one monomer chosen from acrylamides and methacrylamides substituted on the nitrogen with an alkyl radical,

b) at least one acidic comonomer comprising at least one reactive carboxylic groups, and

c) at least one basic comonomer such as esters comprising substituents chosen from primary, secondary, tertiary and quaternary amine substituents of acrylic and methacrylic acids and the product of quaternization of dimethylaminoethyl methacrylate with dimethyl or diethyl sulphate.

In one new embodiment, the N-substituted acrylamides or methacrylamides are, for example, groups in which the alkyl radicals comprise from 2 to 12 carbon atoms such as N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen, for example, from acrylic acids, methacrylic acids, crotonic acids, itaconic acids, maleic acids and fumaric acids and alkyl monoesters, comprising 1 to 4 carbon atoms, of maleic or fumaric acids or anhydrides.

The basic comonomers are chosen, for example, from aminoethyl, butylaminoethyl, N,N′-dimethylaminoethyl and N-tert-butylaminoethyl methacrylates.

The copolymers having the CTFA (4th edition, 1991) name octylacrylamide/acrylates/butylaminoethyl methacrylate copolymer such as the products sold under the name Amphomer or Lovocryl 47 by the company National Starch can be used.

(3) crosslinked and alkylated polyamino amides partially or totally derived from polyamino amides of general formula:

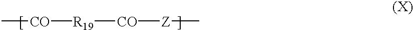

wherein R₁₉ is chosen from divalent radicals derived from a saturated dicarboxylic acid, mono- and dicarboxylic aliphatic acids comprising an ethylenic double bond, an ester of a lower alkanol, comprising 1 to 6 carbon atoms, of these acids and a radical derived from the addition of any one of the acids to an amine chosen from bis(primary) and bis(secondary) amines, and Z is chosen from a bis(primary), mono- and bis(secondary) polyalkylene-polyamine radicals and, for example, Z represents:

a) in proportions ranging from 60 to 100 mol %, the radical of formula (XI):

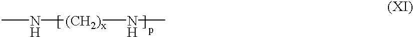

wherein x=2 and p=2 or 3, or x=3 and p=2, this radical being derived from a compound chosen from diethylenetriamine, triethylenetetraamine and dipropylenetriamine;

b) in proportions ranging from 0 to 40 mol %, the radical (XI) above in which x=2 and p=1 and which is derived from a compound chosen from ethylenediamine and piperazine:

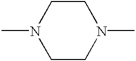

c) in proportions ranging from 0 to 20 mol %, the —NH—(CH₂)₆—NH— radical, which is derived from hexamethylenediamine, these polyamino amides can be crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

In one embodiment, the saturated carboxylic acids are chosen from acids comprising 6 to 10 carbon atoms, such as adipic acid, 2,2,4-trimethyladipic acid and 2,4,4-trimethyladipic acid, terephthalic acid and acids comprising an ethylenic double bond such as, for example, acrylic acid, methacrylic acid and itaconic acid.

The alkane sultones used in the alkylation are chosen, for example, from propane sultone and butane sultone, and the salts of the alkylating agents can be chosen, for example, from the sodium and potassium salts.

(4) polymers comprising zwitterionic units of formula:

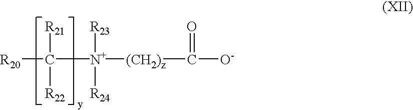

wherein R₂₀ is chosen from a polymerizable unsaturated groups such as acrylate, methacrylate, acrylamide and methacrylamide group, y and z, which may be identical or different, are chosen from integers ranging from 1 to 3, R₂, and R₂₂, which may be identical or different, are chosen from a hydrogen atom, methyl, ethyl and propyl groups, R₂₃ and R₂₄, which may be identical or different, are chosen from a hydrogen atom and alkyl radicals such that the sum of the carbon atoms in R₂₃ and R₂₄ does not exceed 10.

The polymers comprising such units can also comprise units derived from non-zwitterionic monomers such as monomers chosen from dimethyl and diethylaminoethyl acrylates, methacrylates, alkyl acrylates, acrylamides, methacrylamides, and vinyl acetate.

By way of example, mention may be made of the copolymer of methyl methacrylate/methyl dimethylcarboxymethylammonioethylmethacrylate such as the product sold under the name Diaformer Z301 by the company Sandoz.

(5) polymers derived from chitosan comprising monomer units corresponding to formulae (XIII), (XIV) and (XV) below:

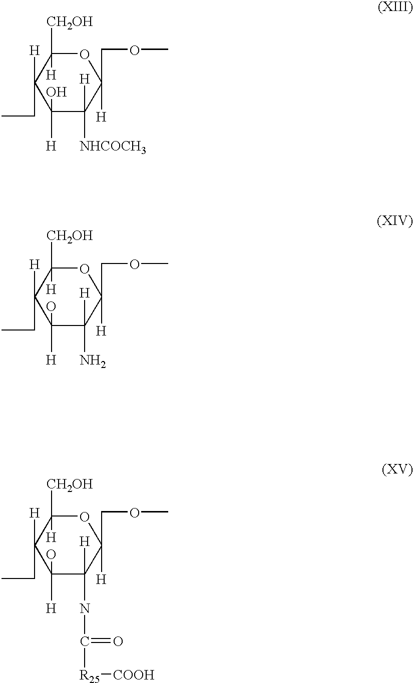

the unit (XIII) being present in proportions ranging from 0 to 30%, the unit (XIV) in proportions ranging from 5% to 50% and the unit (XV) in proportions ranging from 30% to 90%, and wherein in this unit (XV), R₂₅ is a radical of formula:

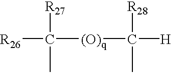

wherein q is chosen from 0 or 1;

if q=0, R₂₆, R₂₇ and R₂₈, which may be identical or different, are each chosen from a hydrogen atom, methyl, hydroxyl, acetoxy and amino residues, and monoalkylamine residues and dialkylamine residues, which are optionally interrupted by at least one nitrogen atom and/or optionally substituted with at least one group chosen from amine, hydroxyl, carboxyl, alkylthio and sulphonic groups, and alkylthio residues in which the alkyl group bears an amino residue, at least one of the radicals R₂₆, R₂₇ and R₂₈ being, in this case, a hydrogen atom;

or, if q=1, R₂₆, R₂₇ and R₂₈, which may be identical or different, are each chosen from a hydrogen atom, and salts formed by these compounds with bases or acids.

(6) polymers derived from the N-carboxyalkylation of chitosan, such as N-carboxymethylchitosan or N-carboxybutylchitosan sold under the name “Evalsan” by the company Jan Dekker.

(7) polymers corresponding to the general formula (XI) as described, for example, in French Patent No. 1 400 366:

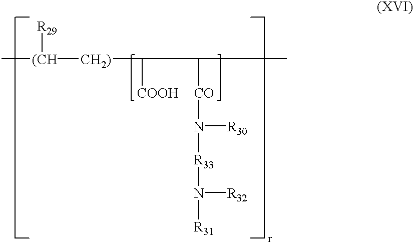

wherein R₂₉ is chosen from a hydrogen atom, and CH₃O, CH₃CH₂O and phenyl radicals, R₃₀ is chosen from a hydrogen atom and lower alkyl radicals such as methyl and ethyl, R₃₁ is chosen from a hydrogen atom and lower alkyl radicals such as methyl and ethyl, R₃₂ is chosen from lower alkyl radicals such as methyl and ethyl and a radicals corresponding to the formula: —R₃₃—N(R₃₁)₂, wherein R₃₃ is chosen from —CH₂—CH₂—, —CH₂—CH₂—CH₂— and —CH₂—CH(CH₃)— groups, and R₃₁ is chosen from a hydrogen atom and lower alkyl radicals such as methyl and ethyl,

and also the higher homologues of these radicals comprising up to 6 carbon atoms, r is chosen such that the number-average molecular weight of said polymer ranges from 500 to 6 000 000, such as from 1000 to 1 000 000.

(8) amphoteric polymers of the type —D—X—D—X— chosen from:

a) polymers obtained by the action of chloroacetic acid or sodium chloroacetate on compounds comprising at least one unit of formula:

—D—X—D—X—D— (XVII)

wherein D is a radical

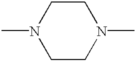

and X is chosen from the symbols E and E′, wherein E and E′, which may be identical or different, are chosen from divalent alkylene radicals comprising at least one chain chosen from straight and branched chains comprising up to 7 carbon atoms in the main chain, wherein the divalent alkylene radicals are optionally substituted with at least one hydroxyl group. E or E′ can additionally comprise at least one atom chosen from oxygen, nitrogen and sulphur atoms, and 1 to 3 rings chosen from aromatic and heterocyclic rings. The

oxygen, nitrogen and sulphur atoms can be present in the form of at least one group chosen from ether, thioether, sulphoxide, sulphone, sulphonium, alkylamine and alkenylamine groups, hydroxyl, benzylamine, amine oxide, quaternary ammonium, amide, imide, alcohol, ester and urethane groups;

b) polymers of formula:

—D—X—D—X— (XVIII)

wherein D is a radical

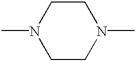

and X is chosen from the symbol E and E′ and wherein at least one X is chosen from E′ ; E having the meaning given above and E′ being a divalent radical chosen from divalent alkylene radicals comprising at least one chain chosen from straight and branched chains comprising up to 7 carbon atoms in the main chain, wherein said divalent alkylene radicals are optionally substituted with at least one hydroxyl radical and comprise at least one nitrogen atom substituted with an alkyl chain, which is optionally interrupted by an oxygen atom, and further comprising at least one functional group chosen from carboxyl functional groups and hydroxyl functional groups which are betainized by reaction with a reactant chosen from chloroacetic acid and sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also comprise other vinyl comonomers such as vinylcaprolactam.

In one embodiment, the amphoteric polymers are chosen from polymers comprising those of family (1).

The concentration of the amphoteric polymer may range from 0.01% to 10% by weight, for example, from 0.05% to 5% by weight and further, for example, from 0.1% to 3% by weight relative to the total weight of the composition.

In another embodiment, the compositions can further comprise at least one surfactant.

The at least one surfactant may be chosen from anionic, amphoteric, nonionic, zwitterionic and cationic surfactants.

The at least one surfactant can be chosen from, for example, the following:

(i) Anionic Surfactants:

By way of example of anionic surfactants which can be used, alone or as mixtures, mention may be made, by way of non-limiting example, of salts (such as alkali metal salts, for example, sodium salts, ammonium salts, amine salts, amino alcohol salts or magnesium salts) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; (C₆-C₂₄)alkyl sulphosuccinates, (C₆-C₂₄)alkyl ether sulphosuccinates, (C₆-C₂₄)alkylamide sulphosuccinates; (C₆-C₂₄)alkyl sulphoacetates; (C₆-C₂₄)acyl sarcosinates; and (C₆-C₂₄)acyl glutamates. It is also possible to use (C₆-C₂₄)alkylpolyglycoside carboxylic esters such as alkylglucoside citrates, alkylpolyglycoside tartrates and alkylpolyglycoside sulphosuccinates, alkylsulphosuccinamates; acyl isethionates and N-acyl taurates. The alkyl or acyl radical of all of these different compounds comprises, for example, from 12 to 20 carbon atoms and the aryl radical is chosen, for example, from phenyl and benzyl groups. Among the anionic surfactants which can be used, mention may also be made, by way of non-limiting examples, fatty acid salts such as oleic, ricinoleic, palmitic and stearic acid salts, coconut oil acid or hydrogenated coconut oil acid and their salts; acyl lactylates wherein the acyl radical comprises from 8 to 20 carbon atoms. It is also possible to use alkyl D-galactoside uronic acids and their salts, polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids and their salts, for example, those comprising from 2 to 50 alkylene oxide groups, such as ethylene oxide groups, and mixtures thereof.

(ii) Nonionic Surfactants:

The nonionic surfactants are compounds that are well known (see, for example, in this respect “Handbook of Surfactants” by M.R. Porter, published by Blackie & Son (Glasgow and London), 1991, pp. 116-178), and their nature is not a critical factor. They can be chosen, for example, from polyethoxylated or polypropoxylated, alkylphenols, alpha-diols and alcohols, comprising a fatty chain comprising, for example, 8 to 18 carbon atoms, it being possible for the number of ethylene oxide or propylene oxide groups to range, for example, from 2 to 50. Mention may also be made of copolymers of ethylene oxide and of propylene oxide, condensates of ethylene oxide and of propylene oxide with fatty alcohols; polyethoxylated fatty amides comprising, for example, from 2 to 30 mol of ethylene oxide, polyglycerolated fatty amides comprising on average from 1 to 5, such as 1.5 to 4, glycerol groups; polyethoxylated fatty amines comprising, for example, from 2 to 30 mol of ethylene oxide; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, and amine oxides such as (C₁₀-C₁₄)alkylamine oxides or N-acylaminopropylmorpholine oxides.

(iii) Amphoteric or Zwitterionic Surfactants:

The amphoteric or zwitterionic surfactants, the nature of which is not a critical factor can be chosen, for example, from aliphatic secondary and tertiary amine derivatives wherein the aliphatic radical is chosen from linear and branched chains comprising 8 to 18 carbon atoms and comprising at least one water-solubilizing anionic group (for example carboxylate, sulphonate, sulphate, phosphate and phosphonate); mention may also be made of (C₈-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines.

Among the amine derivatives, mention may be made of the products sold under the name Miranol, as described in U.S. Pat. Nos. 2,528,378 and 2,781,354 and classified in the CTFA dictionary, 3rd edition, 1982, under the names amphocarboxyglycinates and amphocarboxypropionates, with the respective structures of:

R₂—CONHCH₂CH₂—N⁺(R₃)(R₄)(CH₂COO⁻)

wherein: R₂ is chosen from alkyl radicals derived from an acid R₂—COOH present in hydrolysed coconut oil, and heptyl, nonyl and undecyl radicals, R₃ is a beta-hydroxyethyl group and R₄ is a carboxymethyl group;

and of

R₂—CONHCH₂CH₂—N(B)(C)

wherein:

B represents —CH₂CH₂OX′, C represents —(CH₂)_{z}—Y′, with z=1 or 2,

X′ is chosen from the —CH₂CH₂—COOH group and a hydrogen atom,

Y′ is chosen from the —COOH group and the —CH₂—CHOH—SO₃H radicals,

R′₂ is chosen from alkyl radicals of an acid R′₂—COOH present in coconut oil or in hydrolysed linseed oil, alkyl radicals, such as C₇, C₉, C₁₁, and C₁₃ alkyl radicals, a C₁₇ alkyl radical and its iso form, and an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphopropionate, disodium caprylamphodipropionate, disodium caprylamphodipropionate, lauroamphodipropionic acid and cocoamphodipropionic acid.

By way of example, mention may be made of the cocoamphodiacetate sold under the trade name Miranol® C2M concentrate by the company Rhodia Chimie.

(iv) Cationic surfactants:

The at least one cationic surfactant, may be chosen, for example, from salts of optionally polyoxyalkylenated primary, secondary and tertiary fatty amines; quaternary ammonium salts such as tetraalkylammonium, alkylamidoalkyltrialkylammonium, trialkylbenzylammonium, trialkylhydroxyalkylammonium and alkylpyridinium chlorides and bromides; imidazoline derivatives; and amine oxides of cationic nature.

The amount of the at least one surfactant present in the composition can range, for example, from 0.01% to 40% by weight such as from 0.5% to 30% by weight relative to the total weight of the composition.

The pH of the ready-to-use composition ranges from 1.5 to 12.

The pH of the ready-to-use composition intended for bleaching may range, for example, from 1.5 to 10 and further, for example, from 1.5 to 7.

The pH of the ready-to-use composition intended for permanent reshaping may range from 6 to 12 and further, for example, from 7 to 11.

This pH may be adjusted to the desired value using at least one acidifying agent and/or at least one basifying agent chosen from acidifying agents and basifying agents that are well known in the prior art in the bleaching or permanent reshaping of keratin fibres.

The at least one basifying agent may be chosen from, for example, aqueous ammonia, alkali metals and ammonium carbonates, alkanolamines such as monoethanolamine, diethanolamine and triethanolamine and their derivatives, oxyethylenated and oxypropylenated hydroxyalkylamines and ethylenediamines, sodium hydroxide, potassium hydroxide and the compounds of formula (XIX) below:

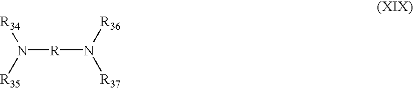

wherein

R is a propylene residue optionally substituted with a group chosen from a hydroxyl group and C₁-C₄ alkyl radicals;

R₃₄, R₃₅, R₃₆ and R₃₇, which may be identical or different, are chosen from hydrogen, C₁-C₄ alkyl radicals, and C₁-C₄ hydroxyalkyl radicals.

The at least one acidifying agent is chosen from, for example, mineral acids and organic acids, such as hydrochloric acid, and orthophosphoric acid, carboxylic acids, such as tartaric acid, citric acid, lactic acid, and sulphonic acids.

The at least one acidifying agent and the at least one basifying agent may each range from about 0.01% to 30% by weight relative to the total weight of the bleaching or permanent-reshaping composition.

The compositions may also comprise sequestering agents such as, for example, ethylenediaminetetraacetic acid (EDTA).

When the compositions comprising the at least one reducing agent and the at least one aminosilicone are in anhydrous form (powder or cream) the compositions may comprise the agents mentioned above in the form of essentially anhydrous solids and/or liquids and at least one additive chosen from the additives mentioned above in the form of essentially anhydrous solids or liquids.

When the medium comprising the at least one reducing agent is aqueous, it may comprise at least one cosmetically acceptable organic solvent chosen from, for example, alcohols such as ethanol, isopropyl alcohol, benzyl alcohol and phenylethyl alcohol, and polyols and polyol ethers, such as, ethylene glycol monomethyl, monoethyl and monobutyl ether, propylene glycol and its ethers, such as, propylene glycol monomethyl ether, butylene glycol, dipropylene glycol, and diethylene glycol alkyl ethers such as diethylene glycol monoethyl ether and monobutyl ether. The at least one solvent may be present in a concentration ranging from about 0.5% to 20% by weight and for example from about 2% to 10% by weight relative to the total weight of the composition.

The bleaching or permanent-reshaping composition may also comprise an effective amount of at least one agent chosen from agents that are known elsewhere in the art of bleaching or permanent reshaping of human keratin fibres, such as hair.

Needless to say, a person skilled in the art will take care to select the optional additional compounds mentioned above, such that at least one advantageous properties intrinsically associated with the composition for bleaching or permanently reshaping keratin fibres is not, or is not substantially, adversely affected by the envisaged additions.

The bleaching process comprises, for example, applying the ready-to-use reducing composition to wet or dry keratin fibres, leaving the composition to act for an action time, for example, ranging from 1 to 60 minutes, and further, for example, from 10 to 45 minutes, rinsing the fibres and then optionally washing them with shampoo, optionally rinsing them again and optionally drying them.

The permanent-reshaping process comprises, for example, applying the ready-to-use reducing composition according to wet or dry keratin fibres, leaving the composition to act for an action time, for example, ranging from 1 to 60 minutes , and further, for example, from 10 to 45 minutes, optionally rinsing the fibres and then applying an oxidizing composition and leaving it to act for an action time ranging from 1 to 20 minutes and further, for example, from 1 to 10 minutes, and then optionally washing the fibres with shampoo, then optionally rinsing them again and optionally drying them.

Mechanical means for placing the keratin fibres under tension may be used before, during and/or after applying the reducing composition, and may be removed before or after rinsing out the oxidizing composition.

Examples of new embodiments described herein are indicated below, without, however, being limiting in nature.

### EXAMPLE 1

The ready-to-use aqueous bleaching composition below was prepared (amounts expressed as grams of active material):

| | | |
|---|---|---|
| Citric acid | | 7.4 |
| Trisodium citrate dihydrate | | 1 |
| Hydroxyethylcellulose | | 1.5 |
| Sodium ascorbate | | 5.7 |
| L-cysteine | | 2 |
| 2-Oxoglutaric acid | | 0.8 |
| Silicone: DC2-8299 ® from the company Dow Corning | | 2 |
| Magnesium sulphate | | 1 |
| Water | qs | 100 |

The above bleaching composition allowed hair to be bleached, giving it a soft, smooth feel, a shiny appearance and good disentangling.

### EXAMPLE 2

The reducing composition below was prepared:

(expressed as grams of active material):

| | | |
|---|---|---|
| Thioglycolic acid | | 9.2 |
| Arginine | | 15 |
| Aqueous ammonia comprising 20% NH₃ | | 1.86 |
| Ammonium carbonate | | 4.5 |
| Cocoylamidopropylbetaine/glyceryl monolaurate (25/5) as an | | 1.3 |
| aqueous 30% solution | | |
| Peptizer | | 0.8 |
| Silicone: DC2-8299 ® from the company Dow Corning | | 2 |
| Isostearyl alcohol (Tego Alkanol 66 sold by the company | | 12 |
| Goldschmidt) | | |
| Sequestering agent | | 0.4 |
| Fragrance | | 0.4 |
| Demineralized water | qs | 100 |

This reducing composition was applied to a lock of wet hair, rolled up beforehand on a curler 9 mm in diameter.

After leaving the composition to act for 10 minutes, it was rinsed out thoroughly with water.

An oxidizing composition (8-volumes aqueous hydrogen peroxide solution of pH 3 was then applied. After leaving it to act for 10 minutes, the lock was again rinsed thoroughly.

The hair was then unrolled from the curler and dried.

The hair had beautiful curls while at the same time being soft, smooth, shiny and easy to disentangle.

What is claimed is:

1. A cosmetic composition for treating human keratin fibres comprising, in a cosmetically acceptable medium:
(i) at least one reducing agent, and
(ii) at least one aminosilicone comprising at least one aminoethylimino(C₄-C₈)alkyl group, said composition being effective for treating human keratin fibres.

2. The composition according to claim 1, wherein the human keratin fibres are hair.

3. The composition according to claim 1, wherein the at least one aminosilicone has the following formula: 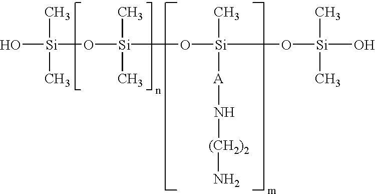
wherein:
A is chosen from linear and branched C₄-C₈ alkylene radicals;
m and n are numbers such that the sum (n+m) ranges from 1 to 2 000; n is a number ranging from 0 to 1 999; and m is a number ranging from 1 to 2 000.

4. The composition according to claim 3, wherein the sum (n+m) ranges from 50 to 150.

5. The composition according to claim 3, wherein the n is a number ranging from 49 to 149.

6. The composition according to claim 3, wherein the m is a number ranging from 1 to 10.

7. The composition according to claim 3, wherein the A is chosen from linear and branched C₄ alkylene radicals.

8. The composition according to claim 1, wherein the viscosity of the at least one aminosilcone is greater than 25 000 mm²/s at 25° C.

9. The composition according to claim 8, wherein the viscosity of the at least one aminosilcone ranges from 30 000 to 200 000 mm²/s at 25° C.

10. The composition according to claim 9, wherein the viscosity of the at least one aminosilicone ranges from 30 000 to 150 000 mm²/s at 25° C.

11. The composition according to claim 1, wherein the at least one aminosilicone has a weight-average molecular mass ranging from 2000 to 1 000 000.

12. The composition according to claim 11, wherein the at least one aminosilicone has a weight-average molecular mass ranging from 3500 to 200 000.

13. The composition according to claim 1, wherein the at least one aminosilicone is in the form of an oil-in-water emulsion comprising at least one surfactant.

14. The composition according to claim 13, wherein the oil-in-water emulsion comprises at least one surfactant chosen from cationic and nonionic surfactants.

15. The composition according to claim 13, wherein the particle size of said at least one aminosilicone ranges from 3 to 500 nanometers.

16. The composition according to claim 15, wherein the particle size of said at least one aminosilicone ranges from 5 to 300 nanometers.

17. The composition according to claim 16, wherein the particle size of said at least one aminosilicone ranges from 10 to 275 nanometers.

18. The composition according to claim 17, wherein the particle size of said at least one aminosilicone ranges from 150 to 275 nanometers.

19. The composition according to claim 1, wherein the at least one aminosilicone comprising at least one aminoethylimino(C₄-C₈)alkyl group is present in the composition in an amount ranging from 0.01% to 20% by weight relative to the total weight of the composition.

20. The composition according to claim 19, wherein the at least one aminosilicone comprising at least one aminoethylimino(C₄-C₈)alkyl group is present in an amount ranging from 0.1% to 15% by weight relative to the total weight of the composition.

21. The composition according to claim 20, wherein the at least one aminosilicone comprising at least one aminoethylimino(C₄-C₈)alkyl group is present in an amount ranging from 0.5% to 10% by weight relative to the total weight of the composition.

22. The composition according to claim 1, wherein the at least one reducing agent is chosen from thiols; thioglycolic acid and thiolactic acid, salts thereof and esters thereof; cysteamine and its salts; sulphites; ascorbic acid, its salts and esters; and erythorbic acid, and its salts and its esters.

23. The composition according to claim 22, wherein the thiols is cysteine.

24. The composition according to claim 1, wherein said at least one reducing agent is present in an amount ranging from 0.1 to 30% by weight relative to the total weight of the composition.

25. The composition according to claim 24, wherein said at least one reducing agent is present in an amount ranging from 0.5% to 20% by weight relative to the total weight of the composition.

26. A process for preparing a composition comprising at least one aminosilicone comprising at least one aminoethylimino(C₄-C₈)alkyl group, said process comprising mixing extemporaneously, at the time of use, an anhydrous composition comprising at least one reducing agent with at least one aqueous composition, wherein at least one of the anhydrous and aqueous compositions comprises said at least one aminosilicone.

27. The composition according to claim 1, wherein the composition is anhydrous and intended for bleaching or permanent reshaping.

28. The composition according to claim 27, wherein the anhydrous composition is in pulverulent form.

29. A process for bleaching or permanently reshaping human keratin fibres, said process comprising:
(A) applying to the fibres a ready-to-use composition comprising, in a medium that is suitable for bleaching, at least one reducing agent and at least one aminosilicone comprising at least one aminoethylimino(C₄-C₈)alkyl group;
(B) leaving said composition to act for an action time ranging from 1 to 60 minutes, optionally rinsing said fibres, optionally washing said fibres with a shampoo, optionally rinsing again, and optionally drying the fibre.

30. The method according to claim 29, wherein said action time ranges from 10 to 45 minutes.

31. The method according to claim 29, further comprising, in a permanent reshaping process, applying to the fibres, optionally after rinsing, an oxidizing composition, and leaving said oxidizing composition to act for an action time ranging from 1 to 20 minutes, optionally followed by washing with shampoo, and then optionally rinsing again and optionally drying again.

32. A multi-compartment device or kit for bleaching or permanently reshaping human keratin fibres, comprising multiple compartments, wherein a first compartment comprises at least one powder or at least one aqueous composition, and a second compartment comprises at least one aqueous composition, wherein at least one of the two compartments comprises at least one reducing agent and at least one of the two compartments comprises at least one aminosilicone comprising at least one aminoethylimino(C₄-C₈)alkyl group.

33. A multi-compartment device or kit according to claim 32, wherein the human keratin fibres are hair.
